(19) United States
(12) Patent Application Publication
(10) Pub. No.: US 2024/0350755 A1
(43) Pub. Date: Oct. 24, 2024

(54) MEDICAL VENTILATOR WITH VENTILATION MODES ADAPTED FOR CARDIAC MASSAGE

(71) Applicant: Air Liquide Medical Systems, Antony (FR)

(72) Inventors: Arnaud LESIMPLE, Antony (FR); Marceau Rigollot, Montrouge (FR); Jean-Christophe Richard, Sevrier (FR); Alexandre Brock, Antony (FR); Cyril Lhuillery, Antony (FR)

(21) Appl. No.: 18/640,351

(22) Filed: Apr. 19, 2024

(30) Foreign Application Priority Data
Apr. 19, 2023 (FR) 2303930

(51) Int. Cl. A61M 16/00; A61M 16/04; A61M 16/06

(52) U.S. Cl. CPC A61M 16/022; A61M 16/0066; A61M 16/04; A61M 16/0605; A61M 2016/0027; A61M 2205/50; A61M 2205/505; A61M 2230/43

(57) ABSTRACT

The invention relates to a medical ventilator (1) configured to provide respiratory assistance to a person in cardiac arrest, comprising a motorized blower (2), a gas circuit (3), and storage means (4) for storing ventilation modes (MV1, MV2) to be implemented during cardiac massage or in the absence of cardiac massage, in particular during an RSCA. Selection means (5; 5.1, 5.2) for selecting a ventilation mode (MV1, MV2) enable a user to select one of the stored ventilation modes, and control means (6) control the motorized blower (2) in response to the user's selection of one of said ventilation modes (MV1, MV2), so as to supply the gas at the desired low and high pressures.

### CROSS REFERENCE TO RELATED APPLICATIONS

This application claims the benefit of priority under 35 U.S.C. § 119 (a) and (b) to French Patent Application No. 2303930, filed Apr. 19, 2023, the entire contents of which are incorporated herein by reference.

### BACKGROUND

The invention relates to a medical ventilator, that is to say an apparatus for artificial ventilation, equipped with a micro-blower, in order to provide respiratory assistance adapted to a person, i.e. a patient, in cardiac arrest during cardiopulmonary resuscitation (CPR).

Cardiac arrest is a common cause of death in humans. The management of cardiac arrest, called cardiopulmonary resuscitation (CPR), is based on the implementation of cardiac massage and, simultaneously, assisted ventilation of the person in cardiac arrest, also called the patient.

Cardiac massage involves alternating compression and decompression of the chest, performed by one or more first responders, e.g. medical personnel. The chest compressions performed during cardiac massage make it possible to compensate for the heart pump, so as to cause the blood to circulate through the various organs, in particular for irrigation of the brain. They must therefore be performed as quickly as possible on the person in cardiac arrest. Quality CPR increases the patient's chances of survival and is defined by international guidelines. Thus, the massage should be performed at a frequency of about 100 to 120 beats per minute with a compression depth of between about 4 and 6 cm.

Furthermore, assisted ventilation must also be delivered to the patient by the medical personnel in order to promote the pulmonary gas exchange with the blood, i.e. the supply of oxygen and removal of CO₂. Ventilation can be provided by means of a manual gas insufflator, i.e. a self-filling balloon with a one-way valve or bag valve mask (BVM), or a medical ventilator, i.e. a respiratory assistance apparatus, serving to dispense air, preferably enriched with O₂, to the patient, especially throughout the cardiac massage.

The delivery of ventilation during CPR is difficult because of the simultaneous application of the chest compressions during the cardiac massage. Conventional medical ventilators are not suitable for this situation and, if used during CPR, they emit alarms and/or malfunction during the chest compressions.

In addition, specific ventilation settings must be implemented in order to deliver effective ventilation. It has in fact been shown that, if poorly performed, ventilation of a patient in cardiac arrest can be deleterious for the circulation generated by the chest compressions and for gas exchange, in particular if it is delivered in an excessive or, conversely, insufficient manner.

Similarly, when the patient is resuscitated by CPR and a resumption of spontaneous cardiac activity (RSCA) takes place, the way in which the patient is ventilated has to be modified so as not to be damaging. Chest compressions are then in fact no longer applied, since the cardiac massage stops being performed, and the patient may show signs of respiratory stress that must be monitored. The fraction of inspired oxygen (FiO₂) that is delivered to the patient must also be adapted to this specific situation, i.e. RSCA. Thus, in practice, it is necessary to have a maximum FiO₂ (i.e. 100%) during cardiac arrest with the application of chest compressions, but in the case of RSCA it is necessary to decrease the FiO₂ in order to avoid hyperoxia, which is potentially damaging to the patient.

However, an RSCA may only be temporary. Thus, during CPR, a patient can fluctuate between phases of cardiac arrest and RSAC in a recurrent manner, each time requiring adaptation of the ventilation delivered and also of the parameters monitored by the ventilator.

It is therefore essential to be able to ventilate patients in cardiac arrest autonomously, safely and in a protective manner, hence to be able to adapt the ventilation of the patient according to the situation encountered, that is to say cardiac arrest or RSCA.

EP3636307 proposes a medical ventilator comprising means for selecting the patient category and compatible ventilation modes, including a cardiopulmonary resuscitation (CPR) mode. In this mode, it is possible to select the levels or values of high pressure, expiratory pressure (PEEP), FiPO₂, respiratory frequency or similar. However, said document gives no guidance on how to select these levels in such a way as to ensure effective ventilation in CPR mode.

Furthermore, EP3218037 and EP3323457 propose a medical ventilator on which the medical personnel are able to change the respiratory settings to be used in cases of cardiac massage or in the absence/cessation of cardiac massage. To do this, it is possible to select ventilation modes recorded in the ventilator that are based on parameters (e.g. high and low pressures, frequency) applicable in the case of cardiac massage or, conversely, in the absence of cardiac massage. However, in practice, the proposed settings have proven unsuitable in certain cases, particularly in the RSCA phase, leading in particular to undesirable activation of alarms.

The present invention therefore aims to provide a medical ventilator for artificially ventilating a person, i.e. a patient, in cardiac arrest during CPR, which medical ventilator is improved, particularly over those described by EP3218037 and EP3323457, in such a way as to improve patient ventilation while avoiding or minimizing the triggering of inappropriate alarms.

### SUMMARY

A solution according to the invention therefore relates to a medical ventilator, i.e. a respiratory assistance apparatus, configured to provide respiratory assistance to a person in cardiac arrest, i.e. a patient, comprising:
a motorized (micro) blower configured to supply a respiratory gas,
an internal gas circuit comprising an inspiratory branch configured to convey the gas flow supplied by the motorized blower,
storage means for storing ventilation modes (MV1, MV2), configured to store a plurality of ventilation modes including at least:
a first ventilation mode (MV1) to be implemented during cardiac massage, characterized by first low pressure (PB1) and first high pressure (PH1) values, with PH1>PB1, and
a second ventilation mode (MV2) to be implemented in the absence of cardiac massage, characterized by second low pressure (PB2) and second high pressure (PH2) values, with: PH2>PB2,
selection means for selecting a ventilation mode (MV1, MV2), enabling a user to select at least the first (MV1) or the second (MV2) stored ventilation mode, and
control means configured to control the motorized blower in response to the user's selection of one of said ventilation modes (MV1, MV2), so as to supply the gas at the low and high pressures (PB1, PH1; PB2, PH2) corresponding to the selected ventilation mode (MV1, MV2).

According to the invention, the second stored ventilation mode (MV2) is characterized by a second high pressure value (PH2), such that: PH2<PH1.

In fact, by studying the problems encountered with the type of ventilator described by EP3218037 and EP3323457, the inventors of the present invention found that the proposed configuration was unsuitable since in the RSCA phase, that is to say after interruption of the cardiac massage, hence in the absence of cardiac massage, the patient may himself generate spontaneous ventilation, in addition to the ventilation delivered by the ventilator.

However, according to these prior documents, during the RSCA phase, that is to say in the absence of/after cessation of cardiac massage, it is recommended to deliver a second high pressure, called PH2, higher than the first high pressure, called PH1, used in the cardiac arrest phase, that is to say during cardiac massage.

Increasing this second high pressure PH2 after the cardiac massage therefore poses a problem, because this second high pressure PH2 higher than the pressure PH1, implemented during the massage, can be superimposed on the spontaneous ventilation generated by the patient during the RSCA, potentially generating inappropriate activation of alarms at the ventilator.

Conversely, these problems can be avoided by parameterizing in the ventilator a second high pressure value (PH2), according to the invention, lower than the first high pressure PH1. Moreover, while the application of chest compressions during the cardiac arrest phase, that is to say during cardiac massage, tends to reduce the delivered volumes and therefore justifies the application of a high pressure PH1, this reduction in volumes no longer exists after cessation of the massage, and it is then desirable to lower the applied pressure, i.e. PH2.

Depending on the embodiment considered, the ventilator of the invention may comprise one or more of the following features:
the first low pressure (PB1) is between 0 and 10 cmH₂O, preferably between 0 and 5 cmH₂O.
the first high pressure (PH1) is between 10 and 40 cmH₂O, preferably between 20 and 30 cmH₂O.
the second low pressure (PB2) is between 0 and 20 cmH₂O, preferably between 5 and 10 cmH₂O.
the second high pressure (PH2) is between 10 and 40 cmH₂O, preferably between 15 and 25 cmH₂O.
the first ventilation mode (MV1) is further characterized by a first given frequency (F1) and the second ventilation mode (MV2) is further characterized by a second given frequency (F2), where F2>F1.
the first frequency (F1) is between 5 and 20 cycles/min, preferably between 8 and 12 cycles/min.
the second frequency (F2) is between 5 and 35 cycles/min, preferably between 15 and 20 cycles/min.
it comprises a touch-panel display screen displaying at least one virtual key.
the means for selecting the ventilation mode comprise said at least one virtual key.
the display screen displays in colour or in black and white, preferably in colour.
it further comprises pressure-measuring means arranged in the gas circuit in order to carry out one or more pressure measurements therein.
the pressure-measuring means cooperate with the control means in order to supply them with the pressure measurement(s).
the pressure-measuring means are electrically connected to the control means.
the pressure-measuring means comprise a pressure sensor or pressure sensors.
the control means process the pressure measurement(s) from the pressure sensor(s) in order to determine the pressure in the gas circuit.
the control means process the pressure measurement(s) in order to deliver the adjusted pressure, i.e. PB1, PB2, PH1 and PH2.
the control means comprise at least one microprocessor.
said at least one microprocessor is arranged on an electronic card.
the means for storing ventilation modes (MV1, MV2) comprise at least one computer memory, in particular a flash memory or the like.
the blower comprises an electric motor.
the gas circuit comprises an inspiratory branch and an expiratory branch.
the inspiratory and expiratory branches are connected to each other at a junction piece, such as a Y-piece.
the gas circuit, in particular the inspiratory branch and the expiratory branch, comprises one or more gas passages, such as one or more gas ducts, in particular flexible pipes or the like.
the expiratory branch is configured to collect the patient's exhaled gas (i.e. CO₂-rich gas) and to discharge it to the atmosphere via a gas outlet orifice.
the expiratory branch comprises an expiratory valve, preferably a solenoid valve, controlled by the control means.
it comprises a CO₂ content measurement device for measuring the content (i.e. quantity) of CO₂ in the gas leaving the lungs of the patient, in particular a capnometer.
the CO₂ content measurement device cooperates with the control means in order to provide them with the CO₂ content measurements.
the control means are configured to process the CO₂ content measurements and to order a display, on the display screen, of a CO₂ content curve over time.
the CO₂ content measurement device is arranged on the expiratory branch.
the CO₂ content measurement device is electrically connected to the control means.
it comprises electrical power supply means, in particular a rechargeable battery or a connection to the mains (110/220V), supplying in particular the control means, the electric motor of the blower and the display screen.
the ventilator comprises an outer casing forming a rigid shell.
depending on the case, the first and second low pressures PB, PB2 may be such that: PB1=PB2, PB1<PB2 or PB1>PB2.
advantageously, the first and second low pressures (PB, PB2) are such that: PB1=PB2.
the CO₂ content measurement device is a capnometer.
the second ventilation mode (MV2) is characterized by second low pressure (PB2) and second high pressure (PH2) values corresponding to a ventilation to be implemented after detection of an RSCA of the patient.
the display screen is configured to display the CO₂ content curve over time, and the user selects the second ventilation mode (MV2), via the selection means, after visualization (i.e. after appearance) of a patient's RSCA on the displayed CO₂ content curve over time.
a patient's RSCA is characterized, on the CO₂ content curve over time, by a notable increase in the quantity of CO₂ leaving the lungs of the patient P.
the selection of the second ventilation mode (MV2) by the user is performed by pressing a finger (typically the index finger) on the virtual key displayed (i.e. touch key) on the touch-panel display screen.

### BRIEF DESCRIPTION OF THE DRAWINGS

The invention will now be better understood from the following detailed description, given by way of illustration but without limitation, with reference to the appended figures, in which:

FIG. 1 shows schematically the pressure levels implemented by the medical ventilator of FIG. 2 in the presence or in the absence of cardiac massage.

FIG. 2 shows schematically a medical ventilator according to the invention.

FIG. 3 shows schematically a CO₂ curve over time (in min) showing an RSCA.

### DETAILED DESCRIPTION OF PREFERRED EMBODIMENTS

FIG. 2 shows schematically an embodiment of a respiratory assistance apparatus or ventilator 1 according to the invention, making it possible to provide respiratory assistance adapted to a person, i.e. a patient P, in cardiac arrest, during the implementation of cardiopulmonary resuscitation (CPR) performed by one or more first responders, typically medical personnel such as a doctor, nurse or the like.

It makes it possible to provide suitable mechanical ventilation during or after cardiac massage, i.e. to cover all CPR steps, including the cardiac massage phase(s) comprising chest compressions and decompressions, and the phase(s) of absence or cessation of massage, in particular during a resumption of spontaneous cardiac activity (RSCA) in the patient P, to improve ventilation by avoiding interrupted ventilation, due to inappropriate alarms, and/or unsuitable ventilation, especially excessive ventilation during the phase(s) of cessation of cardiac massage, especially during an RSCA.

The medical ventilator 1 comprises a motorized (micro) blower 2, also called a turbine or compressor, as a gas source delivering a flow of respiratory assistance gas, that is to say a respiratory gas, typically a flow of air or of oxygen-enriched air.

The motorized blower 2 supplies the respiratory gas to a gas circuit 3, such as one or more internal gas passages or ducts, comprising an inspiratory branch 3.1 configured to convey the gas flow, supplied by the motorized blower 2, to the patient P. In other words, the gas circuit 3 makes it possible to fluidically connect the motorized blower 2 to the airways of the patient P, by way of a respiratory interface 10, for example a breathing mask, a tracheal intubation tube or the like.

Conventionally, a motorized blower 2 comprises an electric motor arranged in a protective casing and controlled by the control means 5 of the ventilator 1, which motor makes it possible to set a motor shaft in rotation, which motor shaft carries an impeller serving to aspirate the gas and supply it to the gas circuit 3. The impeller is usually arranged in the internal compartment of a volute above the protective housing for the motor. Typically, the motor may be of the brushless type. The rotation speeds of the motor can be between 500 and 35,000 rpm.

The gas circuit 3 here also comprises an expiratory branch 3.2 designed to collect the gases exhaled by the patient P, which are rich in CO₂, and to discharge them to the atmosphere via a gas outlet orifice 3.21. The expiratory branch 3.1 can comprise an expiratory flow sensor (not shown), for example a hot-wire sensor, electrically connected to the control means 6, and also an expiratory valve 3.22, such as a solenoid valve, controlled by the control means 6. The inspiratory branch 3.1 and expiratory branch 3.2 are fluidically connected to each other at a junction element 3.3, such as a Y-piece, situated upstream of the respiratory interface 10.

The gas circuit 3 also comprises measuring means making it possible to measure at least one parameter representative of the gas flow, chosen from among the pressure of the gas, the flow rate of insufflated gas, the flow rate of gas exhaled by the patient P and the speed of rotation of the micro-blower, and to deliver at least one signal (or value), representative of said at least one measured parameter, to the control means 6, so as to be used there in particular to control the blower 2 or the like. Preferably, at least one pressure sensor 7 is arranged to measure the pressure of the gas in the gas circuit 3, typically in the inspiratory branch 3.1. The measurements (signal or value) are transmitted to the control means 6, which process them.

Furthermore, there is also provided a CO₂ content measurement device 9, in particular a capnometer or the like, making it possible to measure the CO₂ content in the gas leaving the patient's lungs, in order to be able in particular to detect an RSCA.

The transmission of the signal(s) (or values) coming from the measuring means and from the CO₂ content measurement device, i.e. from the capnometer, to the control means 6 takes place via suitable connections, i.e. electrical connections, such as cables or the like.

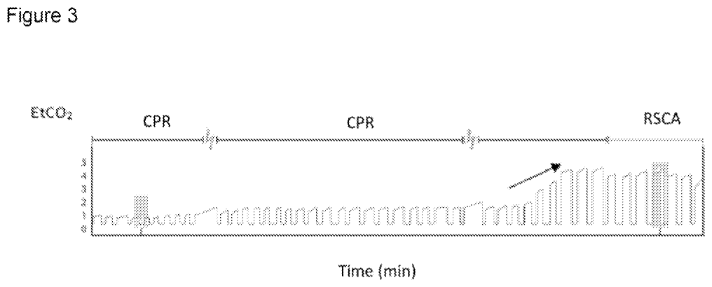

The control means 6 process the CO₂ content measurements and then order a display, on the display screen 8, of a CO₂ content curve over time, that is to say variations of the CO₂ concentration present in the flows leaving the lungs of the patient P, which allows the medical personnel to determine an RSCA, which is characterized by a notable increase in the quantity of CO₂ leaving the lungs of the patient P, as is illustrated in FIG. 3, which shows schematically the quantity of measured CO₂ (EtCO₂) coming from the patient's lungs over time (minutes), i.e. during CPR, and then at the time of resumption of spontaneous cardiac activity or RSCA.

Furthermore, the control means 6 of the ventilator 1 can in particular deduce or determine, from all or some of the transmitted signals, various items of information, in particular a cardiac massage in progress, a volume of gas injected into the patient P, a volume of gas exhaled by the patient P.

The control means 6 of the ventilator 1 comprise one or more electronic cards 6.1 carrying here one or more microprocessors 6.2 programmed in particular with one or more processing algorithms for processing all or some of the various signals received, in particular for performing calculations and comparisons, for establishing tracking curves or other curves, and/or for acting in response to all or some of these signals or to their processing.

Storage means 4, such as one or more flash memories or the like, are also provided, in particular for recording ventilation modes MV1, MV2.

Indeed, given that the levels of high pressure, low pressure and ventilation frequency in particular, delivered during a cardiac massage phase, are different from those delivered during a non-cardiac massage phase, for example following an interruption of massage due to an RSCA of the patient, it is necessary to store various ventilation modes MV1, MV2, which are used by the control means 6 to control the ventilator 1, in particular to control the blower 2 delivering the respiratory assistance gas.

The switch from a first ventilation mode MV1 to a second ventilation mode MV2 is effected by actuation by a user, such as a first responder, i.e. medical personnel, of means 5 for selecting the ventilation mode, which are arranged on the ventilator 1, making it possible to select the first (MV1) or the second (MV2) ventilation mode which are stored in storage means 4.

To do this, a human-machine interface or HMI is provided here comprising a display screen 8, preferably with a touch-sensitive panel, that is to say a digital screen, making it possible to display useful information relating to the ventilation delivered, in particular the ventilation settings, for example the high and low pressure levels, the respiration frequency, the fraction of inspired oxygen, the insufflation time, but also the monitoring of various ventilation parameters, e.g. the insufflation volume, the exhaled volume, the CO₂ content value, the maximum pressure value reached during insufflation, the minute volume, and also curves of various ventilation signals, for example a curve of the CO₂ content of the expired gas, an airway flow curve, an airway pressure curve, a volume curve, etc.

The HMI further comprises the selection means 5, for example push buttons or rotary buttons, cursors, activation or selection keys or the like, enabling the user to make choices, adjustments, selections, confirmations or the like.

The selection means 5 also make it possible, if necessary, to modify the mechanical ventilation parameters proposed automatically by the ventilator 1, or even to indicate to the ventilator 1 a change in the nature of the gas used, for example the change from air to an air/oxygen mixture or a change in the oxygen content of an air/oxygen mixture.

Advantageously, the selection means 5 comprise one or more virtual keys 5.1, 5.2 displayed on the touch-panel display screen 8, so that the user can select the desired first (MV1) or second (MV2) ventilation mode by pressing a virtual key 5.1, 5.2.

The contact of the user's finger on the virtual key(s) 5.1, 5.2 displayed on the touch-panel display screen 8 is recognized by the ventilator 1, and a corresponding signal is transmitted to the control means 6 of the ventilator 1, typically to the microprocessor 6.2 carried by the electronic card 6.1. These can then retrieve the parameters of the first (MV1) or second (MV2) ventilation mode selected within the storage means 4, in particular the pairs of high and low pressure values (PB1, PH1; PB2, PH2), and can use them to control in particular the blower 2, so that the latter delivers the respiratory gas at the desired high and low pressure levels.

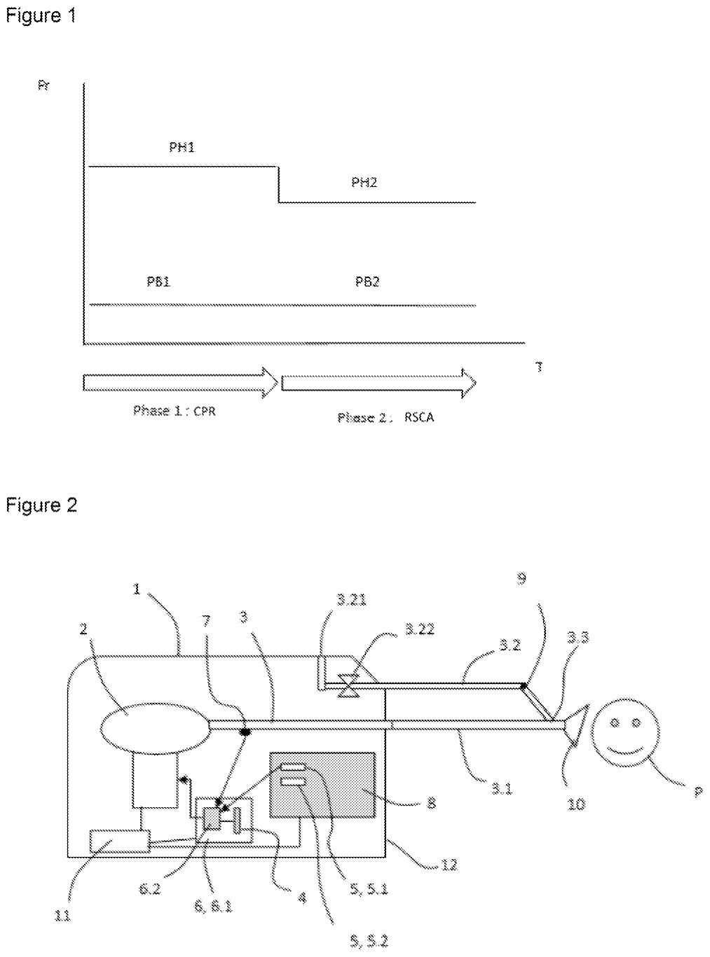

For example, as is illustrated in FIG. 1, representing the pressures (Pr) implemented over time (T), at the start of CPR with cardiac massage and assisted ventilation of a patient in cardiac arrest, the user will press the virtual key 5.1 displayed on the touch-panel display screen 8 of the ventilator 1 in order to signal to the control means 6 of the ventilator 1 that CPR has started, hence it must launch a first ventilation mode (MV1) characterized by first low PB1 and high PH1 pressures corresponding to pressures to be implemented during the cardiac massage phase (phase 1: CPR), for example a first low pressure PB1 of 5 cmH₂O and a first high pressure PH1 of 20 cmH₂O.

The patient is then ventilated between these two pressure levels throughout the cardiac massage phase (phase 1), during which the patient is subjected to alternating chest compressions and decompressions (not detailed in FIG. 1).

Then, for example after several minutes of cardiac massage (phase 1), an RSCA of the patient may be observed, as is illustrated in FIG. 3. At this moment, the user, i.e. the medical personnel, will press the virtual key 5.2 displayed on the touch-panel display screen 8 in order to signal to the control means 6 of the ventilator 1 that CPR has stopped, that is to say that cardiac massage is no longer in progress, and therefore that it must launch a second ventilation mode (MV2) characterized by second low PB2 and high PH2 pressures corresponding to pressures to be implemented in the absence of cardiac massage (phase 2), for example a second low pressure PB2 of 5 cmH₂O and a second high pressure PH2 of 15 cmH₂O. Here, the low pressures PB1, PB2 are equal, that is to say PB2=PB1; however, they could be different.

In other words, the selection of the second ventilation mode MV2 by the user is performed via the selection means 5; 5.1, 5.2, after visualization of the patient's RSCA appearing on the displayed CO₂ content curve, i.e. a notable increase in the quantity of CO₂ leaving the lungs of patient P, as can be seen in FIG. 3 (i.e. arrow).

According to the invention, it is essential that PH2 be less than PH1. It has in fact been demonstrated, within the context of the present invention, that in the RSCA phase, that is to say after interruption of cardiac massage, i.e. in the absence of cardiac massage, the patient may generate spontaneous ventilation himself and that it is then necessary to modulate the ventilation delivered by the ventilator 1 in order to avoid problems of activation of inappropriate alarms. In addition, in the cardiac arrest phase, cardiac massage tends to reduce the volumes delivered by the application of a high pressure PH1. After cessation of massage, this reduction in the volumes no longer exists, and it is then desirable to lower the applied pressure, i.e. PH2, such that PH2<PH1.

Furthermore, when switching from the first ventilation mode (MV1) to the second ventilation mode (MV2), the low pressure may, depending on the circumstances, be kept constant, i.e. PB1=PB2, or increased, i.e. PB2>PB1, or alternatively decreased, i.e. PB2<PB1.

In addition, the ventilation frequency, i.e. the alternating high and low pressures implemented, may also vary when switching from the first ventilation mode (MV1) to the second ventilation mode (MV2), for example it may be increased in the event of cessation of cardiac massage in order to compensate for the loss of ventilation caused by the cessation of the chest compressions of cardiac massage, for example the ventilation frequency can increase from an initial frequency F1 of the order of 10 cycles/min to a higher frequency F2 of the order of 15 cycles/min. Conversely, the frequency can go back from F2 to F1 if chest compressions are restarted in the event of a new cardiac arrest.

In other words, the frequency F2 implemented in the absence/cessation of cardiac massage (MW2 mode), typically during an RSCA, and the frequency F1 implemented during cardiac massage (MW1 mode) are such that: F2>F1.

Typically, F1 is between 8 and 12 cycles/min and F2 is between 15 and 20 cycles/min.

By analogy, the fraction of inspired oxygen (FiO₂) can also be decreased if cessation of cardiac massage is detected, typically during an RSCA, for example the FiO₂ delivered can be 50%. Conversely, FiO₂ can be increased in the event of resumption of chest compressions, for example in the event of a new cardiac arrest after an RSCA, and can then go back from 50% to 100%.

In general, the components of the ventilator 1, in particular the blower 2, the control means 5, at least part of the gas circuit 3, and the display screen 8, are arranged in a casing 12 forming a rigid shell of the medical ventilator 1.

The ventilator 1 and its components requiring energy to operate, in particular the electric motor of the blower 2, the control means 5, the display screen 8, the sensor or sensors 7, 9, are supplied, directly or indirectly, with electric current (110/220 V) coming from an electric current source 11, that is to say from electrical power supply means, for example one or more rechargeable batteries, a power supply of a rescue vehicle or the mains network. If necessary, the ventilator 1 can also incorporate a current converter designed to lower the supply voltage.

In addition, the medical ventilator 1 of the invention can also have a gas mixer cooperating with flow sensors in such a way as to mix a given flow rate of pure oxygen with a given flow rate of air, so as to deliver an inspired fraction of oxygen adjusted so as to adapt the inspired fraction of oxygen delivered to the patient.

In general, the medical ventilator 1 of the invention is particularly well suited for use in the context of cardiopulmonary resuscitation (CPR) comprising the implementation of cardiac massage and simultaneously of assisted ventilation of the person in cardiac arrest, by means of the medical ventilator 1 of the invention.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as fall within the spirit and broad scope of the appended claims. The present invention may suitably comprise, consist or consist essentially of the elements disclosed and may be practiced in the absence of an element not disclosed. Furthermore, if there is language referring to order, such as first and second, it should be understood in an exemplary sense and not in a limiting sense. For example, it can be recognized by those skilled in the art that certain steps can be combined into a single step.

The singular forms “a”, “an” and “the” include plural referents, unless the context clearly dictates otherwise.

“Comprising” in a claim is an open transitional term which means the subsequently identified claim elements are a nonexclusive listing (i.e., anything else may be additionally included remain within the scope of “comprising”). “Comprising” as used herein may be replaced by the more limited transitional terms “consisting essentially of” and “consisting of” unless otherwise indicated herein.

“Providing” in a claim is defined to mean furnishing, supplying, making available, or preparing something. The step may be performed by any actor in the absence of express language in the claim to the contrary.

Optional or optionally means that the subsequently described event or circumstances may or may not occur. The description includes instances where the event or circumstance occurs and instances where it does not occur.

Ranges may be expressed herein as from about one particular value, and/or to about another particular value. When such a range is expressed, it is to be understood that another embodiment is from the one particular value and/or to the other particular value, along with all combinations within said range.

All references identified herein are each hereby incorporated by reference into this application in their entireties, as well as for the specific information for which each is cited.

What is claimed is:

1. A medical ventilator (1) configured to provide respiratory assistance to a person in cardiac arrest, comprising:
a motorized blower (2) configured to supply a respiratory gas,
a gas circuit (3) comprising an inspiratory branch (3.1) configured to convey the gas flow supplied by the motorized blower (2),
storage memory (4) for storing ventilation modes (MV1, MV2), configured to store a plurality of ventilation modes including at least:
a first ventilation mode (MV1) to be implemented during cardiac massage, characterized by first low pressure (PB1) and first high pressure (PH1) values, with PH1>PB1, and
a second ventilation mode (MV2) to be implemented in the absence of cardiac massage, characterized by second low pressure (PB2) and second high pressure (PH2) values, with PH2>PB2,
selection means (5; 5.1, 5.2) for selecting a ventilation mode (MV1, MV2), enabling a user to select at least the first (MV1) or the second (MV2) stored ventilation mode, and
control system (6) configured to control the motorized blower (2) in response to the user's selection of one of said ventilation modes (MV1, MV2), so as to supply the gas at the low and high pressures (PB1, PH1; PB2, PH2) corresponding to the selected ventilation mode (MV1, MV2),
characterized in that the stored second ventilation mode (MV2) is characterized by a second high pressure value (PH2) such that:
PH2<PH1.

2. The ventilator according to claim 1, characterized in that:
the first low pressure (PB1) is between 0 and 10 cmH₂O, preferably between 0 and 5 cmH₂O;
the first high pressure (PH1) is between 10 and 40 cmH₂O, preferably between 20 and 30 cmH₂O;
the second low pressure (PB2) is between 0 and 20 cmH₂O, preferably between 5 and 10 cmH₂O; and/or
the second high pressure (PH2) is between 10 and 40 cmH₂O, preferably between 15 and 25 cmH₂O.

3. The ventilator according to claim 1, characterized in that the first ventilation mode (MV1) is further characterized by a first given frequency (F1) and the second ventilation mode (MV2) is further characterized by a second given frequency (F2), with F2>F1.

4. The ventilator according to claim 1, characterized in that:
the first frequency (F1) is between 5 and 20 cycles/min and/or
the second frequency (F2) is between 5 and 35 cycles/min.

5. The ventilator according to claim 1, characterized in that the ventilator comprises a touch-panel display screen (8) displaying at least one virtual key (5.1, 5.2) for selecting a ventilation mode.

6. The ventilator according to claim 1, characterized in that the storage memory (4) for storing ventilation modes (MV1, MV2) comprise at least one computer memory.

7. The ventilator according to claim 1, characterized in that the ventilator further comprises pressure-measuring device (7) arranged in the gas circuit (3), in order to carry out one or more pressure measurements therein, and cooperating with the control system (6) in order to supply the control system with the pressure measurement or measurements.

8. The ventilator according to claim 1, characterized in that the control system (6) comprises at least one microprocessor (6.2), and preferably said at least one microprocessor (6.2) is arranged on an electronic card (6.1).

9. The ventilator according to claim 1, characterized in that the ventilator comprises a CO₂ content measurement device (9) for measuring the CO₂ content in the gas leaving the patient's lungs and for transmitting the CO₂ content measurements to the control system (6), and the control system is configured to process said CO₂ content measurements and to produce a display, on a display screen (8), of a CO₂ content curve over time.

10. The ventilator according to claim 1, characterized in that the gas circuit (3) is fluidically connected to a respiratory interface (10), preferably a breathing mask or a tracheal intubation tube.

11. The ventilator according to claim 1, characterized in that the first and second low pressures (PB, PB2) are such that: PB1=PB2.

12. The ventilator according to claim 9, characterized in that the CO₂ content measurement device (9) is a capnometer.

13. The ventilator according to claim 4, characterized in that:
the first frequency (F1) is between 8 and 12 cycles/min, and/or
the second frequency (F2) is between 15 and 20 cycles/min.

14. The ventilator according to claim 1, characterized in that the second ventilation mode (MV2) is characterized by second low pressure (PB2) and second high pressure (PH2) values corresponding to a ventilation to be implemented after detection of an RSCA of the patient.

15. The ventilator according to claim 9, characterized in that:
the display screen (8) is configured to display the CO₂ content curve over time, and
the selection of the second ventilation mode (MV2) by the user is made via the selection means (5; 5.1, 5.2), after visualization of an RSCA of the patient on the displayed CO₂ content curve over time, characterized by a notable increase in the quantity of CO₂ leaving the lungs of the patient P.
